(19) United States
(12) Patent Application Publication
(10) Pub. No.: US 2017/0032705 A1
(43) Pub. Date: Feb. 2, 2017

(54) Simulated Organ

(71) Applicant: Seiko Epson Corporation, Tokyo (JP)

(72) Inventors: Hirokazu Sekino, Chino-shi (JP); Jiro Ito, Hokuto-shi (JP); Takeshi Seto, Shiojiri-shi (JP)

(21) Appl. No.: 15/195,788

(22) Filed: Jun. 28, 2016

(30) Foreign Application Priority Data
Jul. 29, 2015 (JP) 2015-149207

(51) Int. Cl. G09B 23/30; A61B 17/3203

(52) U.S. Cl. CPC G09B 23/30; A61B 17/3203; A61B 2217/005

(57) ABSTRACT

A simulated organ includes a simulated parenchyma that simulates a biological parenchyma cell. The simulated parenchyma has a plurality of colors.

### BACKGROUND

1. Technical Field

The present invention relates to a simulated (biological) organ.

2. Related Art

In the related art, a structure including a puncture unit and a simulated blood vessel is known as an injection practice device (for example, JP-A-2012-203153). The puncture unit includes a simulated tissue layer corresponding to a simulated parenchyma, which simulates a parenchyma, i.e. parenchyma cell (s), of a human body. The simulated blood vessel is arranged so as to penetrate the simulated tissue layer. The simulated tissue layer is configured to include a material to which a skin color pigment is added.

In this related art, the simulated parenchyma (simulated tissue layer) is formed of a single uniform color, such as a skin color. Consequently, when the injection practice device is used in the testing of an excision operation aided by the use of a microscope, light is irregularly reflected due to water contained in the simulated parenchyma. The irregularly reflected light and the uniform color limit visibility (and/or depth perception, e.g. a stereoscopic effect), and sufficient visibility in a depth direction can generally not be achieved when the excision is performed. It is noted, however, that this limitation in visibility is not limited to the use of a microscope in the testing of excisions, but is generally common to testing using simulated organs.

### SUMMARY

An advantage of some aspects of the invention is to improve visibility or a stereoscopic effect so as to improve usability.

The invention can be implemented as the following forms.

(1) An embodiment of the invention provides a simulated biological organ. The simulated organ includes a simulated parenchyma that simulates one or more parenchyma cells. The simulated parenchyma has a plurality of colors. According to the simulated organ in the embodiment, a color difference in the simulated parenchyma can improve visibility or a stereoscopic effect, thereby providing excellent usability.

(2) In the simulated organ according to the embodiment, the simulated parenchyma may have different colors in a depth direction. According to this configuration, the visibility or the stereoscopic effect in the depth direction can be improved, thereby enabling the usability to be further improved.

(3) In the simulated organ according to the embodiment, a plurality of the colors may be provided with a marble pattern. According to the simulated organ of the embodiment with this configuration, the marble pattern can be easily employed by insufficiently mixing a plurality of materials having different colors, thereby providing facilitated manufacturing.

(4) In the simulated organ according to the embodiment, a plurality of the colors may be provided a layer of different colors in a depth direction. According to the simulated organ of the embodiment with this configuration, the different colors in a layer shape appear in the depth direction. Therefore, the visibility or the stereoscopic effect in the depth direction can be further improved.

(5) In the simulated organ according to the embodiment, the simulated organ may further include a simulated blood vessel that simulates a blood vessel. The plurality of colors may be respectively different from a color of the simulated blood vessel. According to the simulated organ of the embodiment with this configuration, the simulated organ can include the simulated parenchyma and the simulated blood vessel. Therefore, simulation accuracy can be improved.

(6) In the simulated organ according to the embodiment, a configuration may be adopted in which the simulated parenchyma can be excised by a liquid ejected from a liquid ejecting apparatus. Using the simulated organ of the embodiment with this configuration can improve the usability of the liquid ejecting apparatus.

Objects of the present invention are also met in a simulated organ having a simulated parenchyma that simulates a biological parenchyma cell, wherein the simulated parenchyma has a plurality of colors.

Preferably, the simulated parenchyma has different colors in a depth direction. The plurality of the colors may be provided in a marble pattern. Alternatively, the plurality of the colors may be provided as layers of different colors in a depth direction.

Further preferably, the simulated organ may also include a simulated blood vessel that simulates a biological blood vessel, wherein the simulated blood vessel is of a color different from any of the plurality of the colors of the simulated parenchyma.

If desired, the simulated parenchyma can be excised by a liquid ejected from a liquid ejecting apparatus.

Additionally, the simulated parenchyma may be constructed of a first material of a first color and a second material of a second color different from the first color. In this case, the first material and second material do not form a homogeneous mixture and remain distinct from each other.

If desired, the first material may be arranged into a first plurality of first layers, each first layer being of the first color. Similarly, the second material may be arranged into a second plurality for second layers, each second being of the second color. The first and second layers may then be arranged as adjoining, alternating layers.

In this approach, the adjoining, alternating layers may be arranged horizontally forming a stack of alternating first and second layers.

Alternatively, the adjoining, alternating layers may be arranged vertically, each spanning from a top of the simulated parenchyma to its bottom.

Further alternatively, the first material and second material may be arranged to form a marble pattern distributed throughout the simulated parenchyma.

It is preferred that the first color be achromatic and the second color be chromatic. For example, the first color may be white and the second color may be a warm color, such as orange.

It is further preferred that the first color and the second color be contrasting colors.

Further preferably, the first color is uniformly distributed throughout the first material, and the second color is uniformly distributed through the second material.

The invention can be implemented in various forms in addition to the above-described configurations. For example, the invention can be implemented as a manufacturing method of the simulated organ.

### BRIEF DESCRIPTION OF THE DRAWINGS

The invention will be described with reference to the accompanying drawings, wherein like numbers reference like elements.

FIG. 1 is a schematic view of a configuration of a liquid ejecting apparatus in accordance with the present invention.

FIGS. 2A and 2B are views of a simulated organ.

FIG. 3 is a process diagram illustrating a manufacturing method of the simulated organ.

FIG. 4 illustrates a process of pouring a first material and a second material.

FIG. 5 illustrates a state after a hole is opened in the simulated organ by using the liquid ejecting apparatus.

FIGS. 6A and 6B illustrate a simulated organ in accordance with an alternate embodiment.

FIGS. 7A and 7B illustrate a simulated organ according to an alternate embodiment.

FIG. 8 illustrates a state after a hole is opened in a simulated organ of one of the alternate embodiments by using a liquid ejecting apparatus.

### DESCRIPTION OF EXEMPLARY EMBODIMENTS

Hereinafter, embodiments according to the invention will be described. First, a liquid ejecting apparatus used for excising a simulated organ according to the embodiments will be described.

### A. First Embodiment

### A-1. Configuration of Liquid Ejecting Apparatus

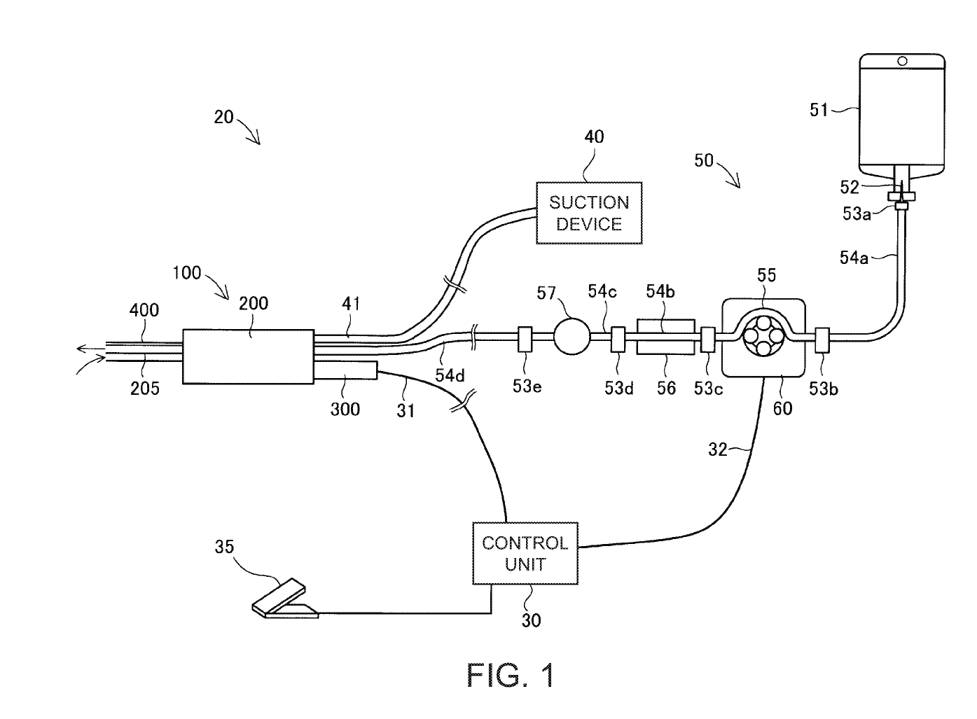

FIG. 1 is a view for schematically describing a configuration of a liquid ejecting apparatus 20. The liquid ejecting apparatus 20 is a medical device used in medical institutions, and is used to excise a lesion by ejecting a liquid toward the lesion.

The liquid ejecting apparatus 20 includes a control unit 30 (i.e. controller), an actuator cable 31, a pump cable 32, a foot switch 35, a suction device (e.g. vacuum) 40, a suction tube 41, a liquid supply device (i.e. liquid supply, or liquid supplier or liquid reservoir) 50, and a handpiece 100.

The liquid supply device 50 includes a water supply bag 51, a spike needle 52, a plurality of connectors (preferably first to fifth connectors 53a to 53e), a plurality of water supply tubes (preferably first to fourth water (or other liquid) supply tubes 54a to 54d), a pump tube 55, a clogging detection mechanism (i.e. clog detector) 56, and a filter 57. The handpiece 100 includes a nozzle unit (i.e. nozzle) 200 and an actuator unit (i.e. actuator) 300. The nozzle unit 200 includes an ejecting tube 205 and a suction pipe 400.

The water supply bag 51 is preferably made of a transparent synthetic resin, and the inside thereof is filled with a liquid (preferably, a physiological saline solution). In the present application, water supply bag 51 is called a “water supply bag” even if it is filled with liquids other than the water. The spike needle 52 is connected to the first water supply tube 54a via the first connector 53a. If the spike needle 52 is stuck into the water supply bag 51, the liquid filling the water supply bag 51 is in a state where the liquid can be supplied to the first water supply tube 54a.

The first water supply tube 54a is connected to the pump tube 55 via the second connector 53b. The pump tube 55 is connected to the second water supply tube 54b via the third connector 53c. The tube pump 60 pinches the pump tube 55. The tube pump 60 feeds (i.e. pumps) the liquid from the first water supply tube 54a side to the second water supply tube 54b side through the pump tube 55.

The clogging detection mechanism 56 detects clogging inside the first to fourth water supply tubes 54a to 54d by measuring pressure inside the second water supply tube 54b.

The second water supply tube 54b is connected to the third water supply tube 54c via the fourth connector 53d. The filter 57 is connected to the third water supply tube 54c. The filter 57 collects foreign substances contained in the liquid.

The third water supply tube 54c is connected to the fourth water supply tube 54d via the fifth connector 53e. The fourth water supply tube 54d is connected to the nozzle unit 200. The liquid supplied through the fourth water supply tube 54d is intermittently ejected from a distal end of the ejecting tube 205 by driving the actuator unit 300. The liquid is intermittently ejected in this way. Accordingly, it is possible to ensure excision capability using a small flow rate.

The ejecting tube 205 and the suction pipe 400 configure a double tube in which the ejecting tube 205 serves as an inner tube and the suction pipe 400 serves as an outer tube. The suction tube 41 is connected to the nozzle unit 200. The suction device 40 applies suction to the inside of the suction pipe 400 through the suction tube 41. The suction is applied to the liquid or excised fragments in the vicinity of the distal end of the suction pipe 400.

The control unit 30 controls the tube pump 60 and the actuator unit 300. Specifically, while the foot switch 35 is stepped on (i.e. actuated or switched on), the control unit 30 transmits drive signals via the actuator cable 31 and the pump cable 32. The drive signal transmitted via the actuator cable 31 drives a piezoelectric element (not illustrated) included (i.e. housed) in the actuator unit 300. The drive signal transmitted via the pump cable 32 drives the tube pump 60. Accordingly, while a user steps on the foot switch 35, the liquid is intermittently ejected. While the user does not step on the foot switch 35, no drive signal is transmitted and liquid ejection is stopped.

### A-2. Configuration of Simulated Organ

Next, a simulated organ according to a first embodiment will be described. The simulated organ is also called a phantom, and is an artificial product whose portion is excised by the liquid ejecting apparatus 20 in the present embodiment. The simulated organ according to the embodiment is used in performing a simulated operation for the purpose of a performance evaluation of the liquid ejecting apparatus 20, manipulation practice of the liquid ejecting apparatus 20, and the like.

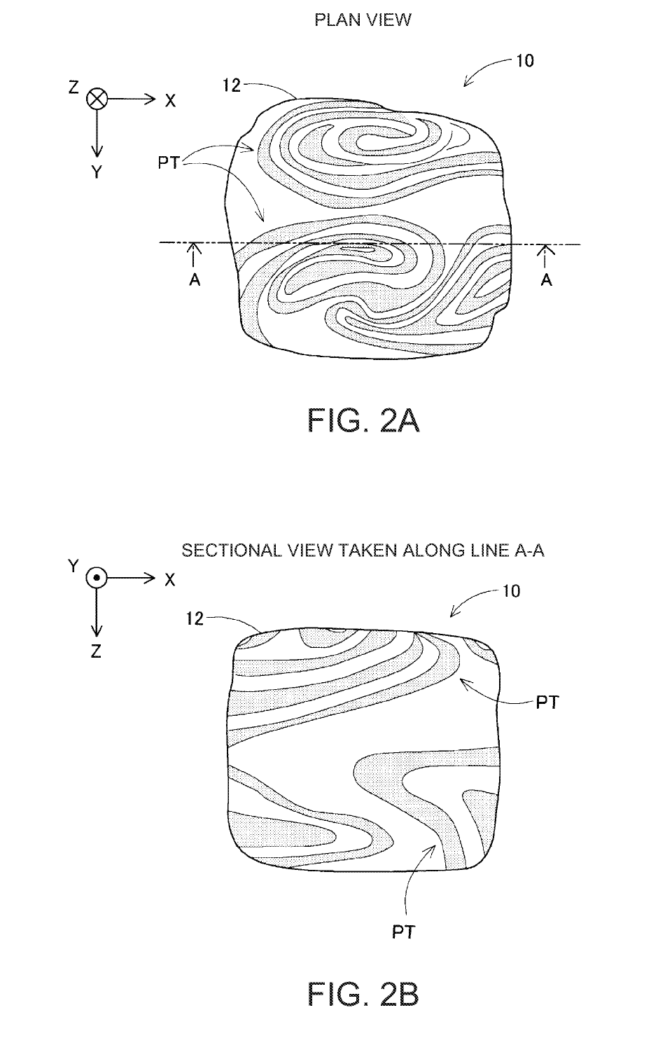

FIGS. 2A and 2B are views for describing a simulated organ 10. FIG. 2A illustrates a plan view, and FIG. 2B illustrates a sectional view taken along line A-A in FIG. 2A. In the embodiment, a horizontal plane represents a plane X-Y, and a vertical direction (i.e., Z-depth direction) represents a direction Z perpendicular to the horizontal plane.

The simulated organ 10 includes a simulated parenchyma 12 and a support member (not illustrated) which supports the simulated parenchyma 12.

The simulated parenchyma 12 is an artificial product that simulates a parenchyma (parenchyma cell(s)) of an organ (i.e. a biological organ such as a human brain, liver, or the like) of a human body. The parenchyma is a cell that directly relates to a characteristic function of an organ. The simulated parenchyma 12 preferably has an externally block shape that is close to a rectangular shape (e.g. roughly resembles a rectangular prism), and is formed with two colors (preferably contrasting or complementary colors, or an achromatic and chromatic combination, or a high contrasting achromatic combination, or a warm/dark color combination, or an achromatic and warm color combination, or a combination of the above color combinations). For example, the two colors may be a white color (achromatic) and an orange color (e.g., a warm, chromatic color). The two colors are in an insufficiently mixed state (e.g. a heterogeneous color mixture). According to the embodiment, the two colors form a marble pattern. In the drawing, a black solid portion is a portion corresponding to the orange color. The marble pattern means a pattern which simulates marble, and appears so that flowing shapes are superimposed on each other or kneaded in a plurality of colors. The simulated parenchyma 12 shows the marble pattern in the horizontal plane direction X-Y as illustrated in FIG. 2A, and also shows the marble pattern in the Z-depth direction, as illustrated in FIG. 2B.

The above-described two colors are not limited to the white color and the orange color. For example, the two colors can be substituted with a combination of various colors, such as the white color and a skin color, the orange color and the skin color, and the like. In addition, without being necessarily limited to the two colors, the number of colors may be three or more. A plurality of the colors indicating two colors, or three or more colors mean a plurality of different colors. However, in the embodiment, the “different colors” mean that a distance between the two colors (difference degree between the two colors in a color space) sufficiently separates the two colors so as to be visible (and preferably easily discriminated) when the two colors are adjacent to each other.

The simulated parenchyma 12 is supported by the support member (not illustrated). The support member may be a metal-based container that accommodates (e.g. holds or cradles) the simulated parenchyma 12 to provide support.

### A-3. Manufacturing Method of Simulated Organ

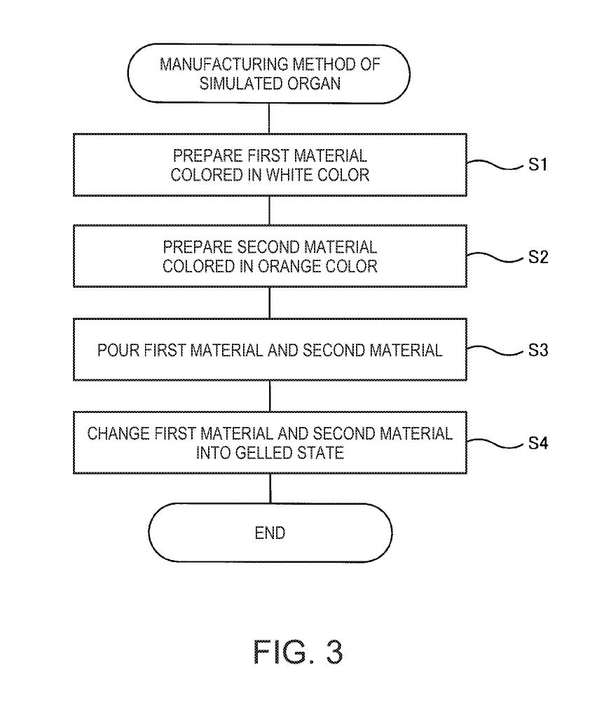

FIG. 3 is a process diagram illustrating a manufacturing method of the simulated organ 10. First, a first material colored in the white color (i.e. a first color) is prepared (Step S1). The embodiment preferably employs polyvinyl alcohol (PVA) as a material of the simulated parenchyma 12. In Step S1, the first material colored with a white colorant (for example, a pigment) mixed with the PVA is prepared.

Next, a second material colored in the orange color (i.e. a second color) is prepared (Step S2). In Step S2, the second material colored with an orange colorant (for example, a pigment) mixed with the PVA is prepared.

Subsequently, the first material prepared in Step S1 and the second material prepared in Step S2 are poured into the container serving as the support member (Step S3).

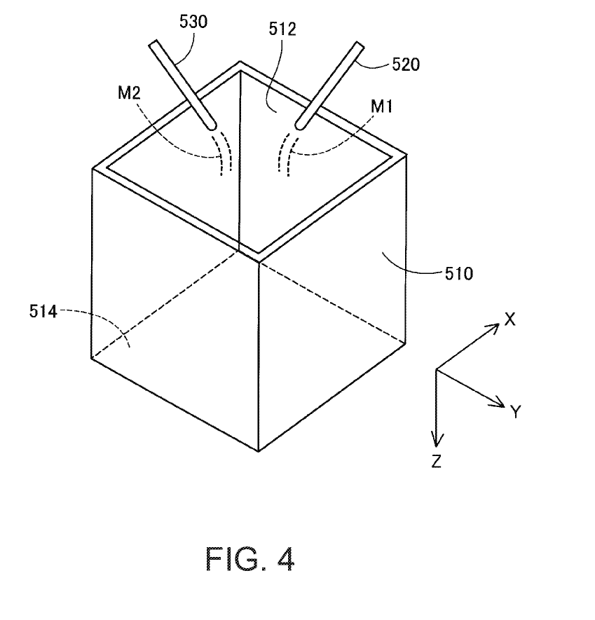

FIG. 4 is a view for describing a state in Step S3. For example, a container 510 has a shape whose upper side has an opening portion 512 and whose lower side has a (sealed) bottom portion 514. A first injection nozzle 520 and a second injection nozzle 530 are arranged above the opening portion 512. A first material M1 prepared in Step S1 is injected from the first injection nozzle 520 toward the opening portion 512. At the same time, a second material M2 prepared in Step S2 is injected from the second injection nozzle 530 toward the opening portion 512. Preferably, the first material M1 does not form a homogenous mixture with the second material M2. As a result, the first and second materials M1 and M2 form the above-described marble pattern.

After Step S3 in FIG. 3, the container 510 into which the first and second materials M1 and M2 are injected is frozen (i.e. subjected to a (preferably cold) temperature treatment at a predefined temperature), thereby changing the first and second materials M1 and M2 so as to be gelled (cured, or solidified) in a mixed state with a marble pattern (Step S4). In this manner, the simulated parenchyma 12 is formed inside the container 510, and the simulated organ 10 is completely manufactured.

### A-4. Advantageous Effect of Embodiment

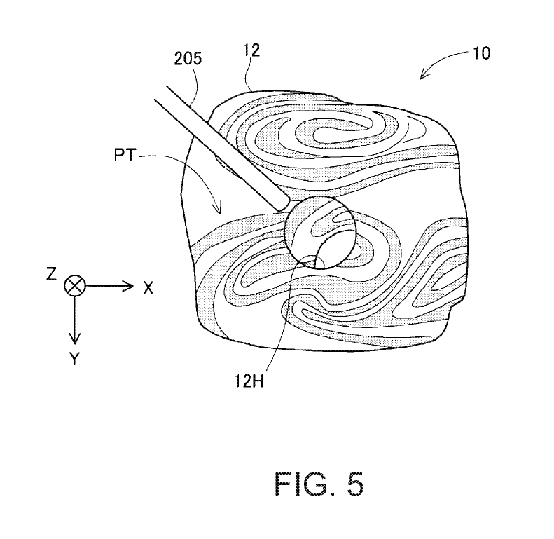

FIG. 5 is a view for describing a state after a hole 12H is opened in the simulated organ 10 by using the liquid ejecting apparatus 20. The simulated organ 10 is illustrated on a plan view. The simulated parenchyma 12 of the simulated organ 10 is gradually excised by a liquid intermittently ejected from the ejecting tube 205 of the liquid ejecting apparatus 20 (FIG. 1), thereby opening the hole 12H in a preferably oblique direction to the Z-depth direction in the drawing. In the simulated organ 10 according to the embodiment, the simulated parenchyma 12 has a marble pattern comprised of two colors. Accordingly, as illustrated in the drawing, the marble pattern also appears on a wall surface of the hole 12H. Therefore, in the simulated organ 10, a color difference provided by the marble pattern can improve visibility or a stereoscopic effect (i.e. depth perception) in the Z-depth direction, thereby providing excellent usability.

In addition, according to the simulated organ 10 in the embodiment, as illustrated in FIG. 4, the marble pattern can be easily formed by injecting the two materials M1 and M2, thereby facilitating manufacturing.

### B. Second Embodiment

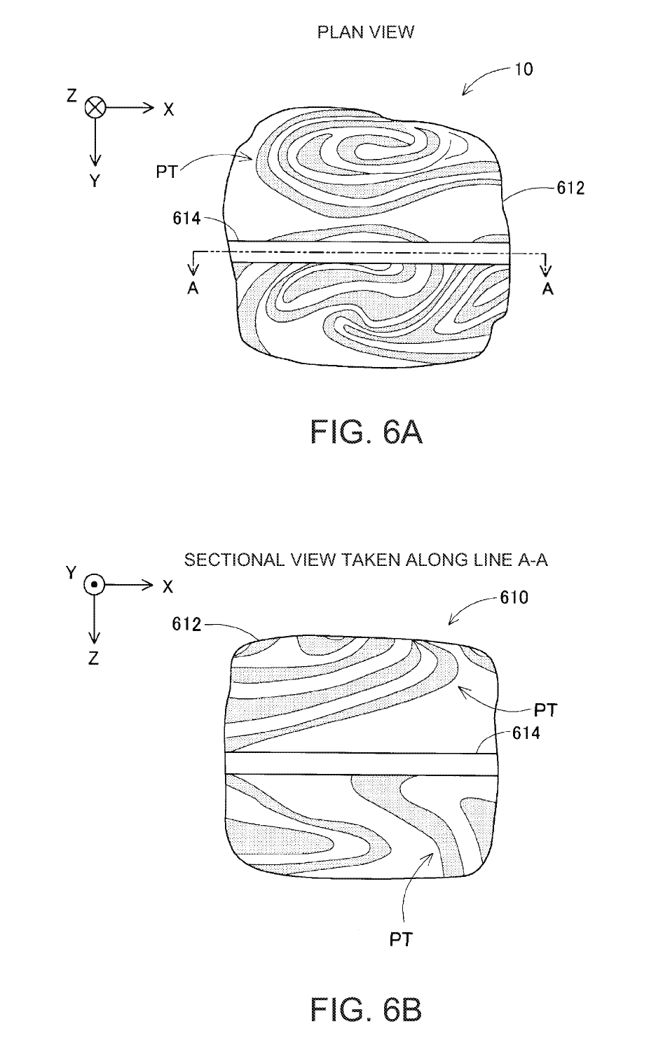

FIGS. 6A and 6B are views for describing a simulated organ 610 according to a second embodiment. FIG. 6A illustrates a plan view of the simulated parenchyma 612 sliced along a horizontal X-Y plane and cutting through a blood vessel 614 within the simulated parenchyma 612. FIG. 6B illustrates a sectional view of the simulated parenchyma 612 sliced along a vertical Z-X plane and cutting through the same blood vessel 614. For illustration purposes, the location of the vertical Z-X plane in FIG. 6B is shown as line A-A in FIG. 6A. FIGS. 6A and 6B would correspond to FIGS. 2A and 2B in the first embodiment.

The simulated organ 610 according to the second embodiment includes a simulated parenchyma 612, a simulated blood vessel 614, and a support member (not illustrated). The simulated parenchyma 612 is similar to the simulated parenchyma 12 included in the simulated organ 10 according to the first embodiment, and has the marble pattern of the white color and the orange color.

The simulated blood vessel 614 is an artificial product that simulates a blood vessel (for example, a human cerebral blood vessel) of a living body, and is formed as a solid member in the embodiment. Polyvinyl alcohol (PVA) may be used in the construction of simulated blood vessel 614. The simulated blood vessel 614 is preferably molded in a red color, and embedded in the simulated parenchyma 612. In the present example, the simulated blood vessel 614 is a member that has to avoid damage in a simulated operation. The simulated blood vessel 614 can be formed as a hollow member in place of a solid member. The color of the simulated blood vessel 614 may be any color other than red, and may be, for example, a blue color. However, the color of the simulated blood vessel 614 is different from the color used for the simulated parenchyma 612.

The support member (not illustrated) is preferably a metal-based container similar to that of the support member according to the first embodiment, and accommodates the simulated parenchyma 612 having the simulated blood vessel 614 embedded therein, thereby supporting the simulated parenchyma 612.

Similarly to the simulated organ 10 according to the first embodiment, in the simulated organ 610 according to the second embodiment configured as described above, a color difference in the simulated parenchyma 612 can improve visibility or a stereoscopic effect in the Z-depth direction, thereby providing excellent usability. In addition, in the simulated organ 610, each color (white and orange) in the simulated parenchyma 612 is different from the color (red) of the simulated blood vessel 614. Accordingly, the visibility of the simulated blood vessel 614 is not impaired. In addition, the simulated organ 610 can be configured to include the simulated parenchyma 612 and the simulated blood vessel 614. Therefore, simulation accuracy can be improved.

According to the second embodiment, the simulated parenchyma 612 employs a white color and an orange color. However, as long as colors different from the color of the simulated blood vessel 614 are used, the parenchyma 612 may be configured using any colors. In addition, without being limited to two, the number of colors used in the construction of the parenchyma 612 may be three or more. In addition, although the second embodiment shows only one simulated blood vessel 614 in the simulated organ 610, two or more simulated blood vessels 614 may be included therein.

### C. Third Embodiment

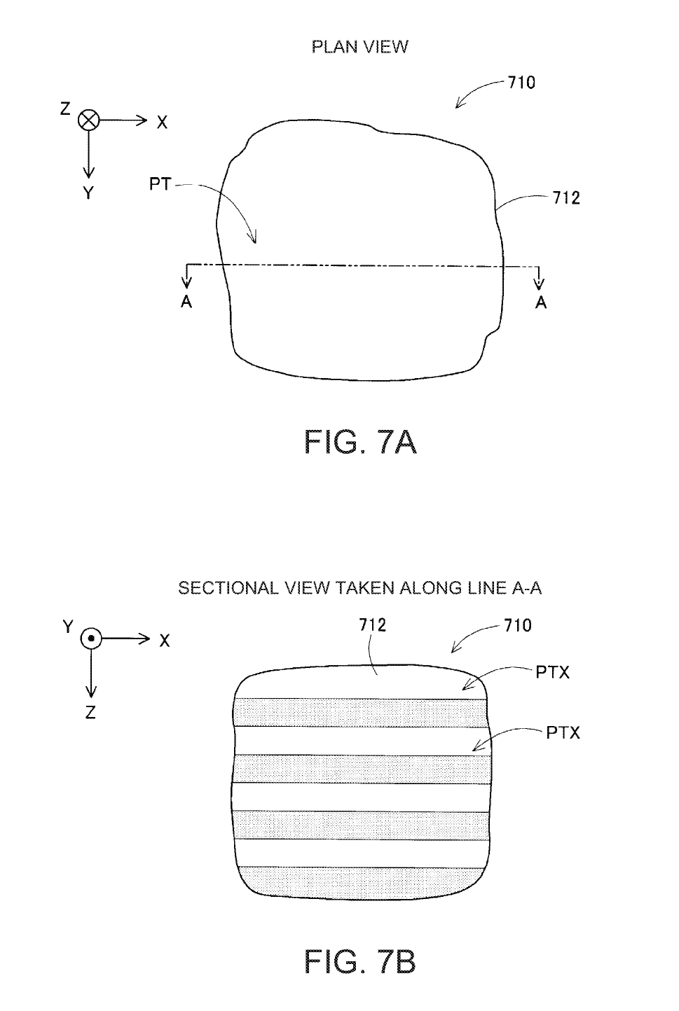

FIGS. 7A and 7B are views for describing a simulated organ 710 according to a third embodiment. FIG. 7A illustrates a plan view, and FIG. 7B illustrates a sectional view taken along line A-A in FIG. 7A. That is, FIGS. 7A and 7B correspond to FIGS. 2A and 2B in the first embodiment.

The first embodiment and the second embodiments, described above, adopt a configuration in which a plurality of colors form a marble pattern in the simulated parenchyma 12 or 612. By contrast in the third embodiment, a simulated parenchyma 712 included in the simulated organ 710 maintains a single color in planes, preferably planes parallel to X-Y planar direction, as illustrated in FIG. 7A, but different planes within simulated parenchyma 712 have different colors. Preferably, alternate layers of planes in the Z-depth direction have alternate colors, as illustrated in FIG. 7B. For example, alternate planar layers may alternate between white and orange. In FIG. 7B, a depicted dark solid linear portion is a portion corresponding to an orange planar layer, and a white linear portion corresponds to a white planar layer. The planar layers are not limited to the two colors The plurality of planar layers may be constructed in a plurality of different colors, such as three or more colors. Without being limited to the combination of the white color and the orange color, the combination of the colors can be substituted with a combination of various colors, such as the white color and the skin color, the orange color and the skin color, and the like.

In a manner similar to the manufacturing method of the simulated organ according to the first embodiment (FIG. 3), the manufacturing method of the simulated organ 710 includes preparing a first material colored in a first color (such as a white color) and a second material colored in a second color (such as an orange color). Next, respectively determined amounts of the first material and the second material are alternately injected into a container (such as container 510) and stacked on each other, thereby forming an alternating layer-shaped pattern.

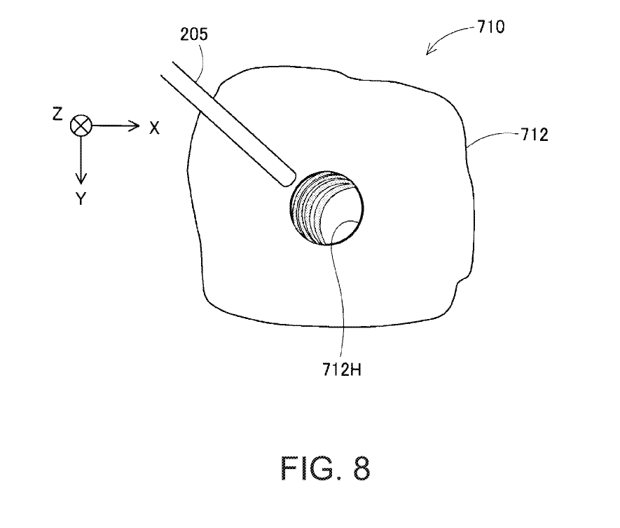

FIG. 8 is a view for describing a state after a hole 712H is opened (i.e. formed) in the simulated organ 710 by using the liquid ejecting apparatus 20. The simulated organ 710 is illustrated on a plan view. The simulated parenchyma 712 of the simulated organ 710 is gradually excised by a liquid intermittently ejected from the ejecting tube 205 of the liquid ejecting apparatus 20 (FIG. 1), thereby opening the hole 712H in a direction oblique to the depth direction Z in the drawing. In the simulated organ 710 according to the embodiment, the simulated parenchyma 712 is formed in the layer shape of different colors in the depth direction Z. Accordingly, as illustrated in the drawing, the layer-shaped pattern also appears on a wall surface of the hole 712H. Therefore, similarly to the first embodiment, in the simulated organ 710 according to the third embodiment, a color difference provided by the layer-shaped pattern can improve visibility or a stereoscopic effect in the depth direction, thereby providing excellent usability.

As a modification example of the third embodiment, a configuration may be adopted so that instead of being comprised of a stack of horizontal layers of alternating colors, the simulated organ 710 may be comprised of a series of adjoined vertical layers of alternating colors. That is, the simulated organ 710 may have a vertical layer formed in a single color in the depth direction Z, and be constructed of multiple such vertical layer shapes of different colors intercepting the plane direction X-Y.

### D. Modification Example

Without being limited to the respective embodiments and modification examples thereof, the invention can be embodied in various forms within the scope of the invention without departing from the invention. For example, the invention can be modified as follows.

### Modification Example 1

In the above embodiments, a primary material in the construction of the simulated parenchyma included in the simulated organ is polyvinyl alcohol (PVA), but the invention is not limited thereto. For example, urethane or a non-urethane, rubber-based (or rubber-like) material may also be used.

### Modification Example 2

The construction material of the simulated blood vessel included in the simulated organ according to the second embodiment is PVA, but the invention is not limited thereto. For example, a non-PVA synthetic resin (for example, urethane) or a natural resin may also be used.

### Modification Example 3

The simulated parenchyma may be manufactured by using injection deposition (e.g. 3D printing using an ink jet method). In addition, the simulated blood vessel may also be manufactured by using 3D printing. Furthermore, the simulated parenchyma and the simulated blood vessel may be collectively manufactured by using 3D printing.

### Modification Example 4

A shape of the simulated parenchyma is configured to be a shape close to a rectangular prism shape, but the invention is not limited thereto. For example, other shapes such as a cylindrical shape, a conical shape, a truncated cone shape, and the like may be used.

### Modification Example 5

The simulated organ may be excised by using methods other than liquid intermittently ejected from a liquid ejecting apparatus. For example, the simulated organ may be excised by using a continuously ejected liquid, or may be excised by using a liquid provided with excision capability using an ultrasound or an optical maser. Alternatively, the simulated organ may be excised by using a metal scalpel.

### Modification Example 6

The above embodiments preferably adopt a configuration in which the piezoelectric element is used as the actuator. However, the embodiments may adopt a configuration in which the liquid is ejected by using an optical maser, a configuration in which the liquid is ejected by a heater generating air bubbles in the liquid, or a configuration in which the liquid is ejected by a pump pressurizing the liquid. According to the configuration in which the liquid is ejected by using an optical maser, the optical maser emits radiation to the liquid so as to generate air bubbles in the liquid, and the resultant increased pressure caused by the generated air bubbles is used to eject the liquid.

Without being limited to the embodiments, the application examples, and the modification examples which are described above, the invention can be implemented according to various configurations without deviating from the scope of present invention. For example, technical features in the embodiments, the application examples, and the modification examples which correspond to technical features according to each embodiment described in the summary of the invention can be appropriately replaced or combined with each other in order to partially or entirely solve the previously described problem or in order to partially or entirely achieve the previously described advantageous effects. If any one of the technical features is not described herein as essential, the technical feature can be appropriately omitted.

What is claimed is:

1. A simulated organ comprising:
a simulated parenchyma that simulates a biological parenchyma cell,
wherein the simulated parenchyma has a plurality of colors.

2. The simulated organ according to claim 1,
wherein the simulated parenchyma has different colors in a depth direction.

3. The simulated organ according to claim 1,
wherein the plurality of the colors are provided in a marble pattern.

4. The simulated organ according to claim 1,
wherein the plurality of the colors are provided as layers of different colors in a depth direction.

5. The simulated organ according to claim 1, further comprising:
a simulated blood vessel that simulates a biological blood vessel,
wherein the simulated blood vessel is of a color different from any of the plurality of the colors of the simulated parenchyma.

6. The simulated organ according to claim 1,
wherein the simulated parenchyma can be excised by a liquid ejected from a liquid ejecting apparatus.

7. The simulated organ according to claim 1, wherein:
the simulated parenchyma is constructed of a first material of a first color and a second material of a second color different from the first color,
the first material and second material not forming a homogeneous mixture and remaining distinct from each other.

8. The simulated organ according to claim 7, wherein:
the first material is arranged into a first plurality of first layers, each first layer being of said first color;
the second material is arranged into a second plurality for second layers, each second being of said second color; and
the first and second layers are arranged as adjoining, alternating layers.

9. The simulated organ according to claim 8, wherein said adjoining, alternating layers are arranged horizontally forming a stack of alternating first and second layers.

10. The simulated organ according to claim 8, wherein said adjoining, alternating layers are arranged vertically, each spanning from a top of the simulated parenchyma to its bottom.

11. The simulated organ according to claim 7, wherein the first material and second material form a marble pattern distributed throughout the simulated parenchyma.

12. The simulated organ according to claim 7, wherein the first color is achromatic and the second color is chromatic.

13. The simulated organ according to claim 12, wherein the first color is white and the second color is a warm color.

14. The simulated organ according to claim 7, wherein the first color and the second color are contrasting colors.

15. The simulated organ according to claim 7, wherein the first color is uniformly distributed throughout said first material, and the second color is uniformly distributed through said second material.
